(19) United States
(12) Patent Application Publication
(10) Pub. No.: US 2026/0294506 A1
(43) Pub. Date: Oct. 1, 2026

(54) SURGICAL BLADE INSTRUMENTS WITH CONDENSABLE VAPOR DELIVERY

(71) Applicant: John H. SHADDUCK, Menlo Park, CA (US)

(72) Inventor: John H. SHADDUCK, Menlo Park, CA (US)

(21) Appl. No.: 19/535,960

(22) Filed: Feb. 10, 2026

Related U.S. Application Data
(60) Provisional application No. 63/779,737, filed on Mar. 28, 2025.

(51) Int. Cl. A61B 18/04; A61B 17/3203; A61B 17/3213; A61B 18/00

(52) U.S. Cl. CPC A61B 18/04; A61B 2017/32035; A61B 17/3213; A61B 2018/00577; A61B 2218/007

(57) ABSTRACT

A vapor-based tissue treatment device comprises a proximal housing carrying a liquid medium source and a pump assembly configured to deliver a controlled flow of liquid medium to a resistive heating structure thermally coupled to a flow channel. The resistive heating structure is configured to convert the liquid medium into a condensable vapor. A working end region includes a blade having a sharp cutting edge and at least one vapor outlet positioned at or near the blade and oriented to emit the condensable vapor toward tissue adjacent the cutting edge to provide coagulation, ablation, or thermal modification during or contemporaneously with cutting. In various embodiments, the blade is fixed or removable relative to a blade support member, and vapor outlets are provided on opposing sides of the blade, within the blade, or through channels defined by the blade support member. The device optionally includes flow stabilization geometries configured to reduce two-phase flow instabilities and improve vapor quality during pulsed vapor delivery.

### CROSS-REFERENCE TO RELATED APPLICATION

This is a non-provisional of U.S. Provisional Application no. 63/779,737 filed Mar. 28, 2025, the entirety of which is incorporated by reference.

### FIELD OF THE DISCLOSURE

The present disclosure relates generally to medical devices, systems, and methods for delivering condensable vapor to tissue, and more particularly to blade-based surgical instruments configured to deliver condensable vapor adjacent a cutting edge for tissue coagulation, ablation, sealing, or modification during or contemporaneously with cutting tissue.

### BACKGROUND OF THE DISCLOSURE

Surgical cutting and coagulation are commonly performed using electrosurgical instruments that apply radiofrequency electrical energy to tissue to achieve cutting, coagulation, or sealing. Such instruments are widely used because they can provide cutting and hemostasis with a single device. However, electrosurgical systems rely on the passage of electrical current through tissue, which may result in non-uniform heating due to variations in tissue properties, contact conditions, and electrode geometry.

The use of electrosurgical energy is associated with a number of recognized challenges and risks. These include unintended thermal injury to adjacent tissue, generation of surgical smoke that may obscure the operative field and raise concerns regarding inhalation exposure, and stimulation of nerves or muscles due to current flow through tissue. In addition, the presence of electrical energy in oxygen-enriched surgical environments introduces a recognized risk of fire, and insulation failures or stray currents may result in unintended burns.

While alternative thermal modalities such as lasers and ultrasound have been developed, such systems may require complex equipment or present other limitations. Accordingly, there remains a need for improved surgical instruments and methods that enable precise cutting with localized tissue coagulation or sealing while reducing reliance on electrical current passing through tissue and mitigating some of the risks associated with conventional electrosurgical devices. The devices and methods disclosed herein address these and other needs by providing blade-based instruments configured to deliver controlled condensable vapor adjacent to a cutting edge during or contemporaneously with cutting.

### SUMMARY OF THE DISCLOSURE

The present invention relates generally to medical devices, systems, and methods for generating and delivering a condensable vapor medium to tissue, and more particularly to blade-based surgical instruments configured to deliver condensable vapor adjacent a sharp cutting edge to achieve localized tissue coagulation, ablation, sealing, desensitization, remodeling, or hemostasis during or contemporaneously with cutting.

In one aspect, the invention provides a vapor ablation device comprising a proximal housing carrying a liquid medium source and a pump assembly configured to deliver a controlled flow of a liquid medium, such as sterile water or saline, to a vapor generation subsystem. The device further includes a working end region comprising a blade having a sharp cutting edge and a blade support member carrying the blade. The blade support member may comprise a rigid carrier, manifold, or cartridge structure configured to support a thin blade element and to cooperate with one or more vapor outlets positioned at or near opposing sides of the blade. In operation, condensable vapor emitted from the vapor outlet(s) is directed toward tissue adjacent to the cutting edge to provide thermal treatment in conjunction with cutting.

In certain embodiments, the blade is fixed relative to the blade support member, while in other embodiments, the blade is removably coupled to the blade support member, such as in a replaceable blade cartridge arrangement. The blade may be formed of any suitable material capable of maintaining a sharp cutting edge, including, without limitation, stainless steel, cobalt-chromium alloys, titanium alloys, ceramic materials, composite structures, coated metals, or combinations thereof.

In various embodiments, vapor outlet and channel architectures may be configured in multiple ways. In one embodiment, vapor is distributed through one or more surface channels formed at an interface between a blade surface and an adjacent surface of the blade support member, wherein the channels terminate in vapor outlet openings oriented toward tissue adjacent the cutting edge. In another embodiment, the blade is a thicker blade member that defines an internal vapor flow channel, with one or more vapor outlets positioned at or immediately adjacent the cutting edge. In still other embodiments, vapor flow channels are formed within the blade support member itself, such that the blade support member functions as a vapor manifold with outlets located on opposing sides of the blade.

In certain embodiments, a single vapor outlet may be provided and oriented toward a divider structure or slot portion of the blade, wherein the divider structure is configured to split the emitted vapor flow such that vapor propagates along both opposing sides of the blade to thermally treat tissue adjacent the cutting edge. Such configurations may provide bilateral vapor delivery while reducing the number of discrete outlet passages required.

In additional embodiments, the working end may include aspiration functionality for the removal of fluids, condensate, smoke, or debris generated during cutting and vapor delivery. In some embodiments, the blade itself defines an aspiration lumen coupled to a negative pressure source, with an aspiration port positioned proximal to at least one vapor outlet. In other embodiments, the blade support member defines an aspiration lumen or channel coupled to a negative pressure source, enabling aspiration functionality with a thin, removable blade insert.

The device further includes a vapor generation subsystem comprising a resistive heating structure thermally coupled to a flow channel configured to convert the controlled flow of liquid medium into a flow of high-quality condensable vapor. In certain embodiments, the heating structure comprises an electrically energized resistive element thermally coupled to metallic tubing defining a heated-wall vaporization region. The heating structure may be positioned within the proximal housing, within an elongate member, near the working end, or in a multi-stage arrangement including a primary vaporization heater and a secondary vapor conditioning heater.

In some embodiments, the vapor generation subsystem includes one or more geometrical flow stabilization features configured to reduce two-phase flow instabilities during vapor generation and to improve vapor quality. Such features may include upstream flow restrictors, downstream expansion zones, staged impedance geometries, diverging vaporization segments, multi-aperture inlet jet structures, internal pin-fin or porous inserts, and manifold or bypass flow passages configured to suppress plug or slug flow transitions and reduce liquid droplet carryover.

In certain embodiments, the device may be configured for fully portable operation, wherein an onboard battery serves as an exclusive electrical power source for the pump assembly, controller, and resistive heating structure during vapor delivery intervals. In further optional embodiments, the device may include coupling features for attachment to a robotic positioning system or robotic arm, while also being usable as a handheld surgical instrument.

In all such embodiments, the disclosed vapor blade platform enables controlled delivery of condensable vapor adjacent to a cutting edge to provide localized thermal treatment with reduced unwanted thermal spread. Variations in blade geometry, vapor outlet architecture, vapor generation configuration, flow stabilization features, aspiration integration, power source, and positioning arrangements are within the scope of the invention.

These and other aspects, embodiments, and advantages of the invention will become apparent from the following detailed description, the accompanying drawings, and the appended claims.

### BRIEF DESCRIPTION OF THE FIGURES

FIG. 1 is a transparent view of a vapor treatment device configured with a proximal housing carrying a vapor-generating module and a working comprising a blade member with at least one vapor outlet at or near the blade configured to direct vapor flow toward the blade edge.

FIG. 2 is an illustration of the device of FIG. 1 coupled to a positioning system, such as a robotic arm assembly, to assist in controlled manipulation, stabilization, or navigation of the working end relative to a target tissue site.

FIG. 3A is an exploded perspective view of a first variation of a blade member and blade support member, wherein the blade is removably carried by the blade support member and configured for vapor delivery along opposing sides of the blade.

FIG. 3B is an assembled perspective view of the variation of FIG. 3A, illustrating a flow channel communicating with surface channels formed at an interface between the blade and the blade support member to deliver vapor to at least one vapor outlet positioned adjacent the blade cutting edge.

FIG. 4 is a perspective view of a second variation of a blade member comprising a thicker blade structure having at least one internal vapor flow channel configured to communicate with one or more vapor outlets positioned at or near the blade cutting edge.

FIG. 5A is a perspective view of a third variation of a blade and blade support member, wherein the blade is fixed or removable, and the blade support member defines vapor flow channels configured to deliver vapor to opposing sides of the blade.

FIG. 5B is an enlarged perspective view of the blade support member of FIG. 5A illustrating vapor flow channels formed in the support member wall and terminating in vapor outlets adjacent opposing blade surfaces.

FIG. 6A is a perspective view of a fourth variation of a blade member having an open slot and divider structure, wherein the blade support member defines a single vapor outlet configured to direct vapor toward the divider structure.

FIG. 6B is an enlarged perspective view of the working end region of FIG. 6A illustrating vapor flow divided by the divider structure to propagate along opposing sides of the blade.

FIG. 6C is a sectional view of the variation of FIGS. 6A-6B illustrating division of vapor flow from a single vapor outlet to opposing sides of the blade.

FIG. 6D is a sectional view of another variation similar to FIGS. 6A-6C, wherein a divider structure extends into the vapor outlet to divide the vapor flow path within the blade support member.

FIG. 7A is a perspective view of a fifth variation of a blade and blade support member wherein the blade support member defines an aspiration port and aspiration channel coupled to a negative pressure source and further includes vapor delivery outlets configured to deliver vapor toward tissue adjacent the blade cutting edge.

FIG. 7B is a sectional view of the variation of FIG. 7A, illustrating an aspiration lumen positioned proximal to at least one vapor outlet to evacuate fluids, vapor, or debris from a treatment site during vapor-assisted cutting.

FIG. 8 is a perspective view of a sixth variation of a blade member comprising a thicker blade structure defining an internal aspiration channel coupled to a negative pressure source, wherein an aspiration port is positioned proximal to at least one vapor outlet.

FIG. 9 is an enlarged view of the proximal housing of FIG. 1 with the cartridge-based motor, pump mechanism, and liquid media syringe in exploded view.

FIG. 10A is an enlarged view of the resistive heating assembly of FIG. 1.

FIG. 10B is an exploded view of the resistive heating assembly of FIG. 10A.

FIG. 11 is a schematic view of a vapor heating assembly including an upstream flow restrictor positioned proximal to a vaporization region of a flow channel, and a downstream expansion zone positioned distal to the vaporization region, wherein the restrictor and expansion zone define a staged-impedance geometry configured to reduce two-phase flow instabilities and improve vapor quality.

FIG. 12 is a schematic view of a vapor heating assembly illustrating a multi-aperture inlet structure positioned proximal to the vaporization region, configured to deliver the liquid medium as multiple inlet jets into the flow channel to improve flow distribution.

FIG. 13 is a schematic view of the vapor heating tube and flow channel illustrating an internal insert positioned within the vaporization region comprising a pin-fin or post array or micro-structured region configured to promote controlled nucleation behavior and mitigate oscillatory two-phase flow during vapor generation.

FIG. 14 is a schematic view of a vapor heating coil illustrating an interconnected manifold or bypass flow passage 350 extending between an upstream junction and a downstream junction relative to the vaporization region, providing an alternate flow path for at least one of liquid and vapor phases to reduce intermittent channel blockage.

### DETAILED DESCRIPTION OF THE DISCLOSURE

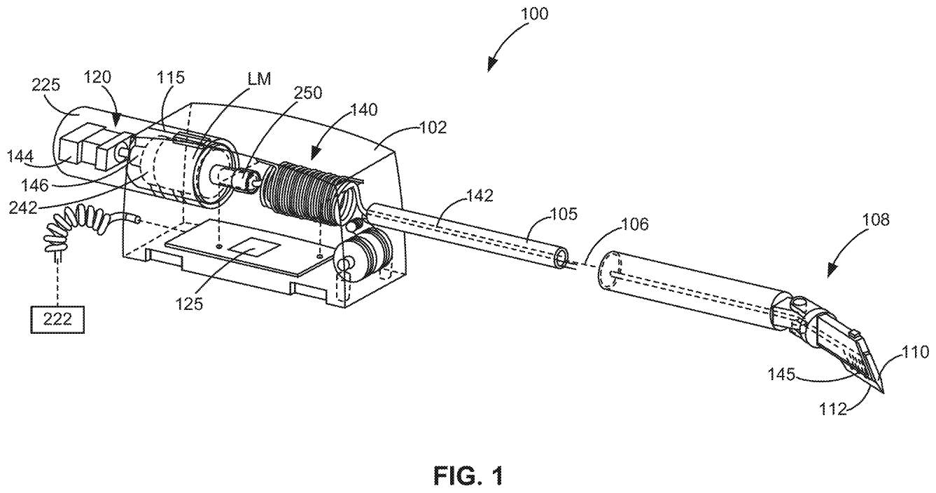

Referring to FIG. 1, a vapor-based tissue treatment device 100 is illustrated in one exemplary embodiment. The device 100 generally comprises a proximal housing 102 or handle, a shaft 105 extending about axis 106, and a working end region 108 including a blade member 110 having a sharp cutting edge 112. In the illustrated embodiment, the proximal housing 102 carries a vapor generation module or subsystem including a liquid medium source or syringe 115, a pump assembly 120, a controller 125, and a resistive heating assembly 140 thermally coupled to a flow channel 142. The vapor generation module is configured to receive a controlled flow of liquid medium LM and convert the liquid medium into a flow of condensable vapor V for delivery distally through the flow channel 142 to at least one vapor outlet 145 located at or near the blade member. As described in greater detail below, the vapor outlet(s) 145 may be configured to direct vapor toward tissue adjacent the cutting edge 112 to provide localized thermal treatment, such as coagulation, ablation, or sealing, during or contemporaneously with cutting. Additional details of the vapor generation subsystem, flow stabilization features, blade support structures, vapor outlet geometries, and optional robotic coupling arrangements are described in the following sections and illustrated in the accompanying figures.

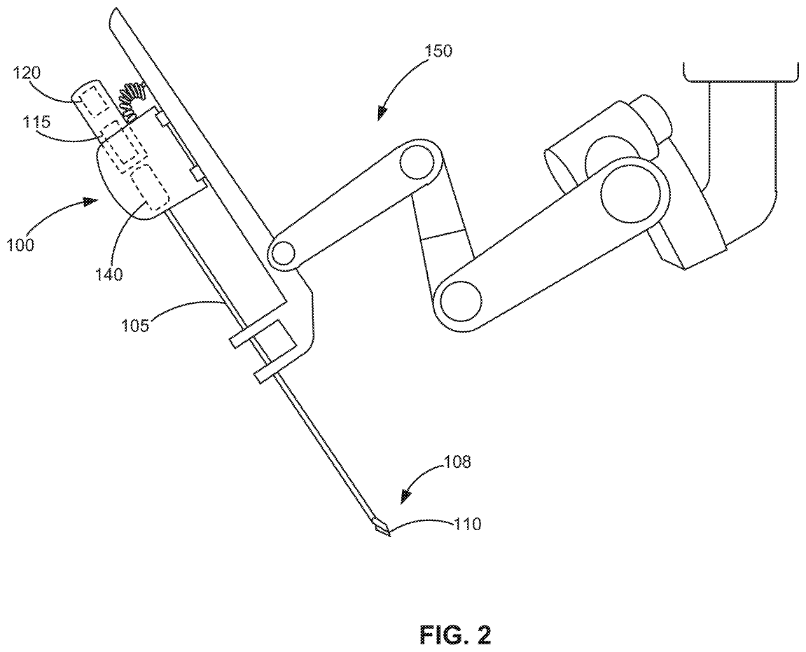

In certain optional embodiments, and as shown schematically in FIG. 2, the device 100 may be configured for coupling to a positioning system, such as a robotic arm assembly 150, to assist in controlled manipulation, stabilization, articulation, or navigation of the working end region 108 relative to a target tissue site. Such robotic coupling may be achieved by any suitable interface, mount, or tool-holding structure associated with the proximal housing 102. In such embodiments, any of the vapor-delivery devices, heating assemblies, delivery interfaces, blades, needles, catheters, and working-end configurations described herein may be configured for coupling to, positioning by, or stabilization by a robotic or computer-assisted positioning system to provide controlled translation, rotation, articulation, and/or advancement of the vapor-delivery instrument relative to patient anatomy. It should be appreciated, however, that robotic positioning is not required in all embodiments, and the device 100 may be used as a fully handheld instrument in a conventional manner with the proximal housing 102 in the form of an ergonomic handle, and thus the robotic coupling arrangement of FIG. 2 is provided as a non-limiting example of an optional accessory or mode of use.

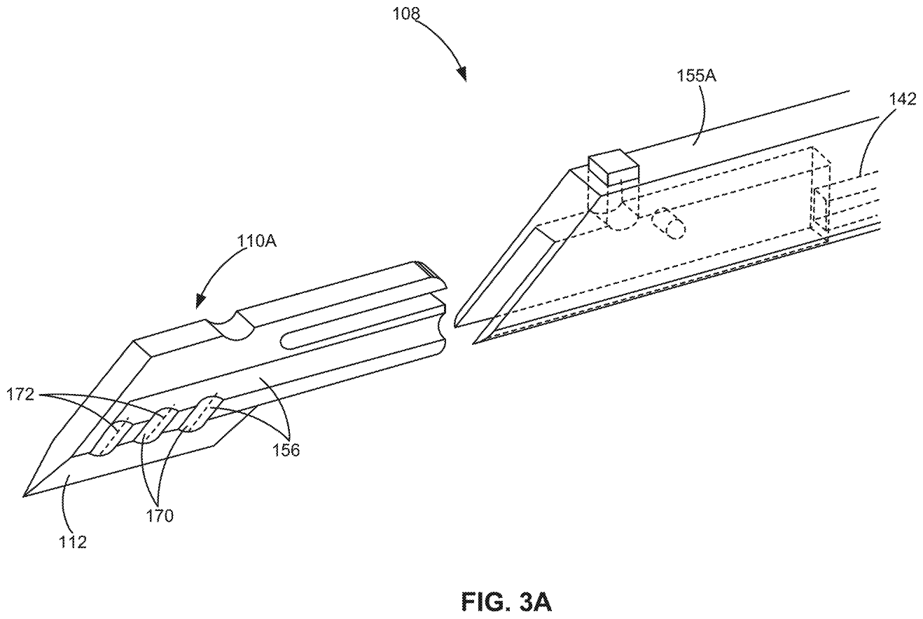

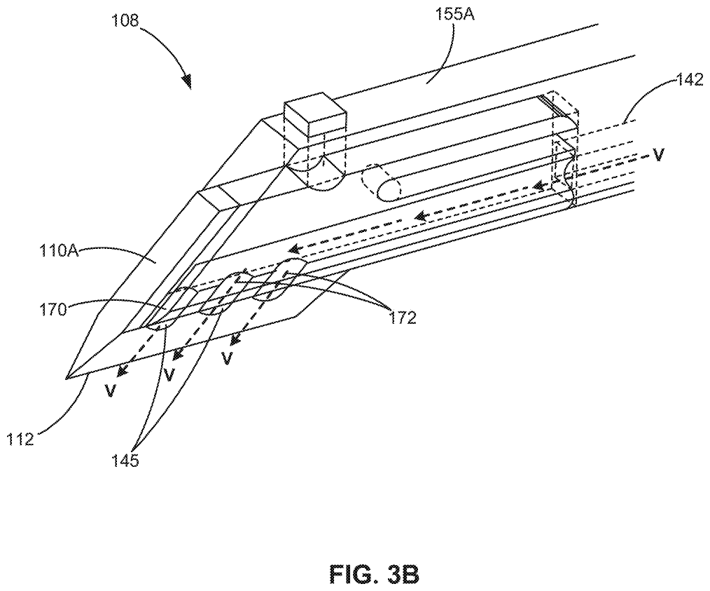

Referring now to FIGS. 3A-8, enlarged schematic views of the working end region 108 illustrate various non-limiting embodiments of a blade-based vapor delivery interface suitable for delivering condensable vapor V adjacent a sharp cutting edge 112. In these embodiments, the working end comprises a blade member having a sharp cutting edge 112 and a blade support member configured to carry, support, or removably retain the blade. The blade support member may further define or cooperate with one or more vapor flow passages and vapor outlet(s) 145 positioned at or near opposing sides of the blade to direct vapor toward tissue adjacent the cutting edge 112. In such embodiments, the blade may comprise any suitable rigid material capable of maintaining a sharp cutting edge, including, without limitation, stainless steel, cobalt-chromium alloys, titanium alloys, ceramic materials, composite materials, coated metals, glass, or combinations thereof. In various embodiments, the blade 110 may be fixed relative to a blade support member, or may be removably coupled thereto, such as by a slot-fit, latching mechanism, cartridge, or other replaceable mounting arrangement as shown below.

As described in greater detail below, the vapor outlet(s) 145 communicate with the flow channel 142 extending from the vapor generation module within the proximal housing 102 (FIG. 1), and may be implemented using surface channels 156, internal blade flow channels or lumens 158, support-member manifold channels 160, and/or divider or slot structures 162 illustrated below. The vapor outlet(s) also may be oriented to cooperate with aspiration features, including aspiration channels 165A, 165B, and aspiration ports 168A, 168B, as shown in various embodiments below. It should be appreciated that the embodiments of FIGS. 3A-8 are provided for purposes of illustration, and that any of the blade configurations, vapor outlet arrangements, flow channel architectures, aspiration features, and stabilization structures described herein may be used individually or in any combination in a given device, consistent with the principles of the invention.

### Blade Variations and Vapor Outlet Configurations

Referring to FIGS. 3A-8, blade members 110A-110E are illustrated as non-limiting examples of blade configurations suitable for use with the vapor blade platform described herein. In each embodiment, one or more vapor outlets 145 are provided at or near the blade edge 112, although the size, shape, and orientation of the vapor outlets may vary between embodiments. The blade members 110A-110E comprise scalpel-type blades configured for cutting while vapor is delivered toward the cutting edge 112.

In a first variation shown in FIGS. 3A and 3B, the blade 110A is fixed in or removably carried in the blade support member 155A with FIG. 3A showing the blade 110A separated from the blade support member. The flow channel 142 extends through the proximal portion of the support member 155A from the resistive heating assembly 140 and communicates with at least one vapor outlet 145 (see FIG. 3B) through one or more surface channels 156 formed in the blade at an interface between both first and second surfaces of the blade 110A and an adjacent surface of the blade support member 155A. Such surface channels 156 may be formed in the surfaces of either or both the blade 110A and the blade support member 155A. In such embodiments, the surface channels 156 extend axially along opposing first and second sides of the blade 110A and terminate in at least one first vapor outlet 145 on a first side of the blade and at least one second vapor outlet 145 on a second side of the blade. The channels are positioned to direct vapor V toward tissue adjacent to the cutting edge 112. In the variation of FIGS. 3A-3B, the surface channels 156 have terminal portions 170 that terminate at each of the first vapor outlet and the second vapor outlet that define an outlet axis 172 oriented to direct emitted condensable vapor V toward the cutting edge 112. FIG. 3B illustrates the assembly of the working end 108, wherein the support member 155A is configured to mechanically support the blade member 110A and provide a suitable seal between the members 110A and 155A so that vapor V is delivered by channels 156 defined by the blade/support interface. As can be seen in FIG. 3B, at least one vapor outlet 145 is spaced apart from the cutting edge, wherein the spaced-apart distance can range from about 1 mm to 5 mm.

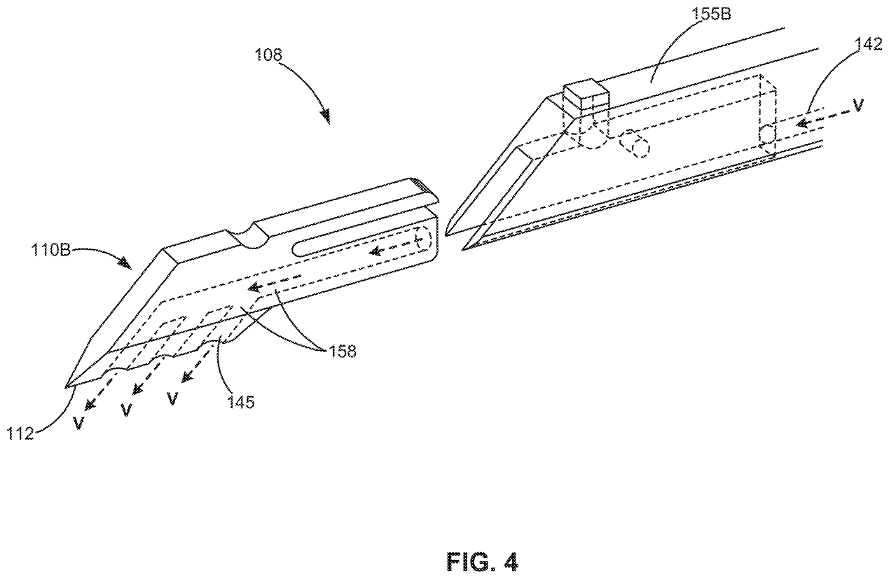

In a second variation shown in FIG. 4, the blade member 110B is thicker and may comprise a ceramic or other rigid material, wherein one or more internal vapor flow channels 158 are formed within the interior of the blade itself. In such embodiments, the internal channel 158 communicates with one or more vapor outlets 145 positioned at or immediately adjacent the sharp cutting edge 112, such that vapor V is emitted directly from the edge region during cutting or coagulation. This configuration may be advantageous when high-pressure vapor flows are used, wherein the vapor V, or water droplets entrained in the emitted vapor flow to thereby apply mechanical forces to assist in cutting tissue in applications where an integrated blade-vapor cutting mechanism is desired.

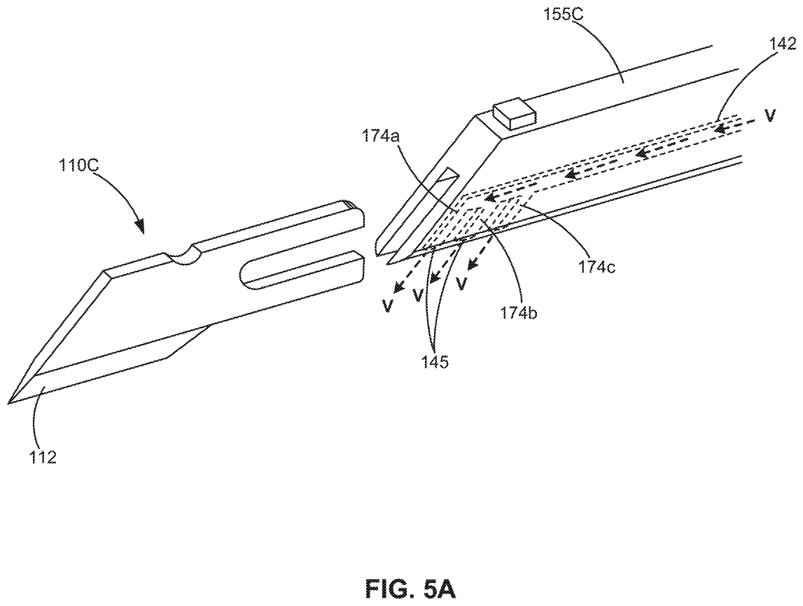

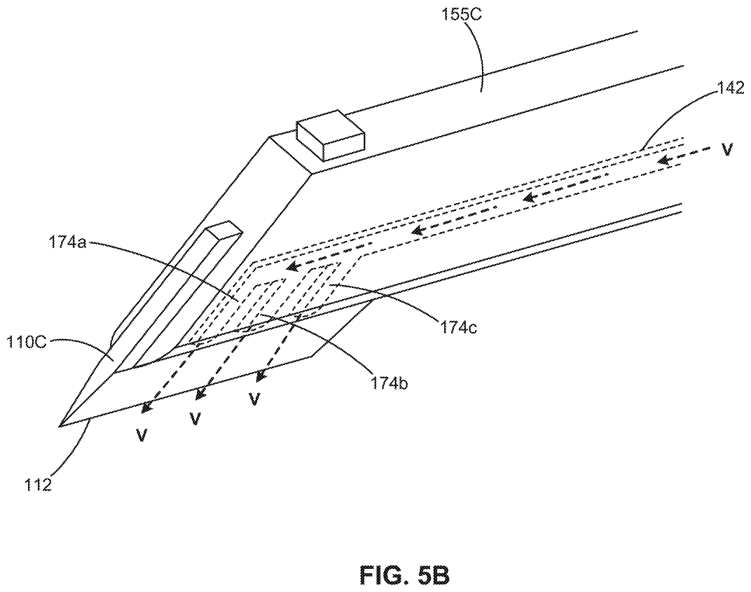

In a third variation shown in FIGS. 5A-5C, the blade 110C is fixed or removable relative to the blade support member 155C, and the flow channel 142 from the resistive heating assembly 140 communicates with one or more channels 174a-174c formed in the wall of the blade support member 155C rather than in the blade interface or the blade itself. In such embodiments, the channels 174a-174c terminate in vapor outlets 145 located on opposing sides of the blade support member 155C adjacent the blade surfaces. This arrangement permits the blade support member 155C to function as a vapor manifold, enabling vapor delivery on both sides of the blade while maintaining a simple blade geometry.

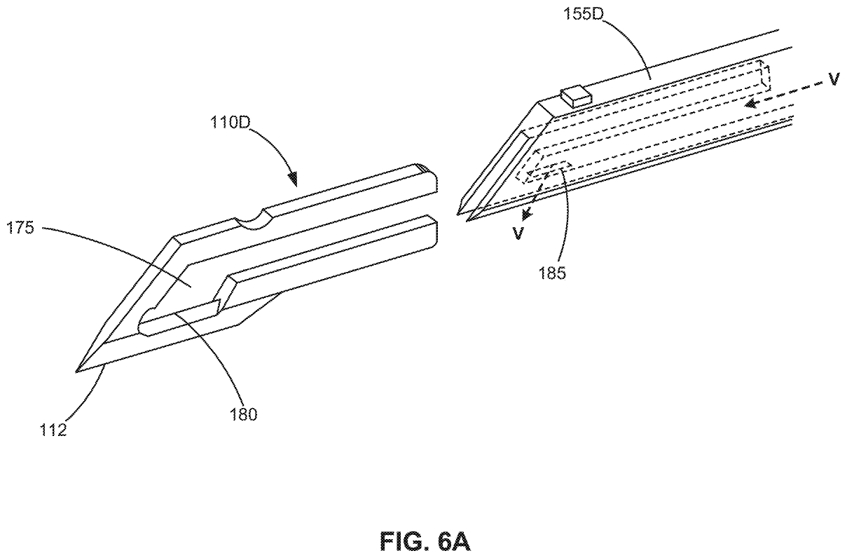

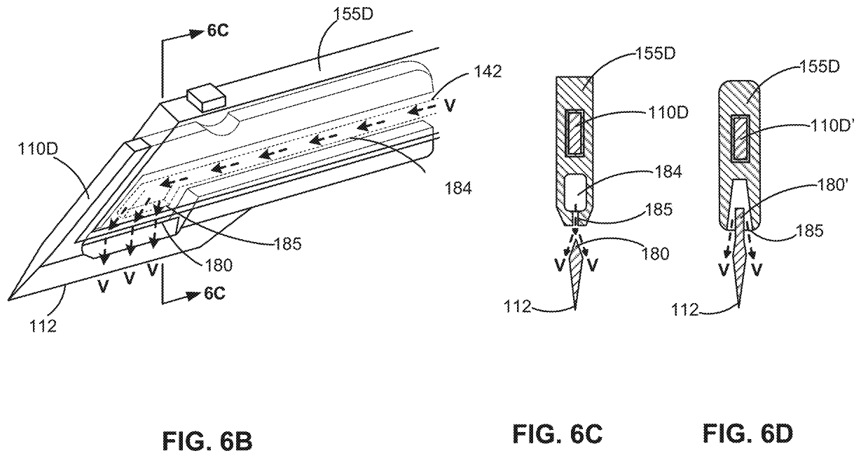

In a fourth variation shown in FIGS. 6A-6C, the blade 110D is fixed or removable and includes an open slot 175 in the blade with a divider structure 180 extending along a superior portion of the slot 175. The blade support member 155D defines a flow channel 184 and a single vapor outlet 185 oriented to emit vapor V toward the divider structure 180, wherein the divider structure is configured to split the emitted flow of vapor V as shown in FIG. 6B and the sectional view of FIG. 6C such that vapor V propagates along both first and second opposing sides of the blade 110D toward the blade edge 112. In such embodiments, a single vapor outlet 185 may provide bilateral tissue treatment adjacent the cutting edge 112 while reducing the number of discrete outlet channels and passages required.

FIG. 6D is a sectional view of another variation similar to that of FIGS. 6A-6C wherein the divider structure 180′ of blade 110D′ extends further into the single vapor outlet 185 in the blade support member 155D′ to further divide the flow path of vapor V inward of the surface of the vapor outlet 185.

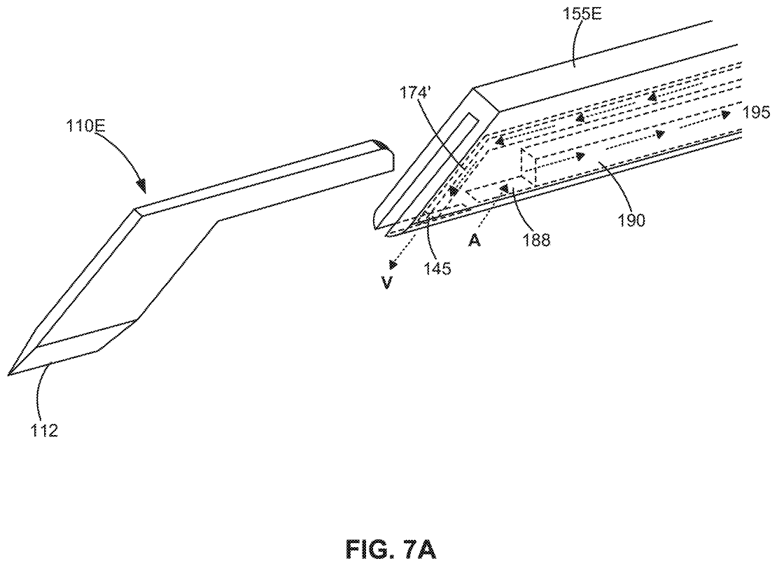

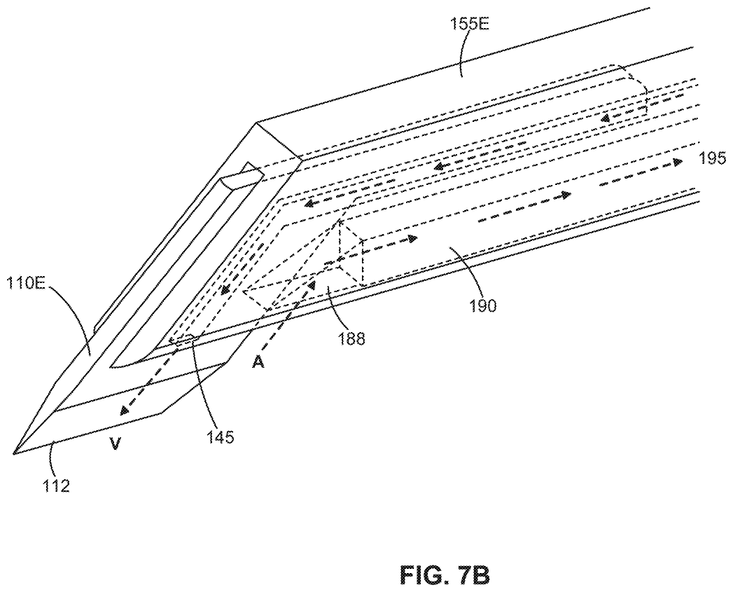

In a fifth variation shown in FIGS. 7A-7B, the blade 110E is fixed or removable relative to the blade support member 155E, and the blade support member defines an aspiration lumen or channel 190 coupled to a remote negative pressure source 195. In such embodiments, an aspiration port 188 and aspiration channel 190 are positioned proximal to at least one vapor outlet 145 to evacuate fluids, vapor, or debris generated during cutting from the treatment site while vapor V is delivered toward tissue adjacent the cutting edge 112. The aspiration channel 190 extends through the support structure 155E to the proximal housing and a collection reservoir. The blade support member 115E of FIGS. 7A-7B has internal vapor inflow channels 174′ as in the variation of FIGS. 5A-5B to deliver vapor V towards the blade edge 112. This configuration may be particularly advantageous where the blade is a thin replaceable insert and aspiration functionality is integrated into the blade support member 155E.

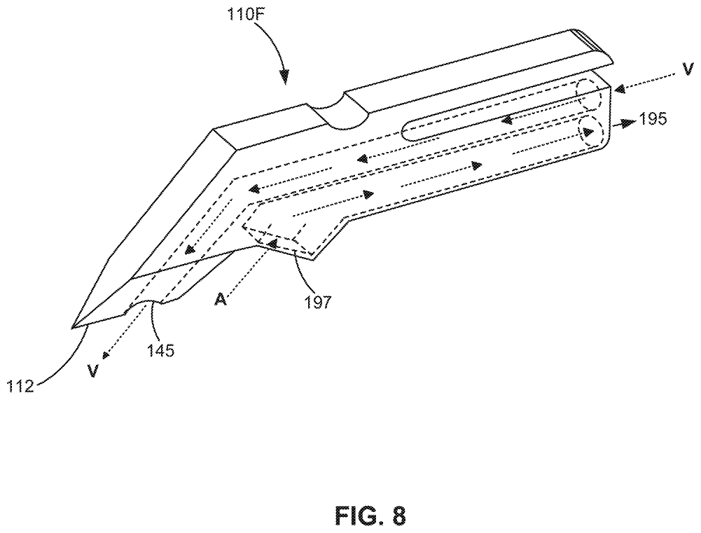

In a sixth variation shown in FIG. 8, the blade member 110F is thicker and defines an aspiration port 197 and an interior aspiration channel 198 that are coupled to a negative pressure source 195 as in the device of FIG. 7B. In such an embodiment, the aspiration port 197 again is positioned proximal to at least one vapor outlet 145 and operates as the blade assembly of FIGS. 7A-7B to aspirate fluids A, condensate, or debris from the treatment site.

It should be appreciated that the blade-based working end configurations of FIGS. 3A to 8 are non-limiting examples, and variations in blade thickness, blade material, vapor channel routing, vapor outlet placement, aspiration integration, and removable blade arrangements are within the scope of the invention. In all such embodiments, the combination of cutting and controlled delivery of condensable vapor V enables simultaneous incision, coagulation, ablation, sealing, or tissue remodeling while reducing unwanted thermal spread.

Further, while FIGS. 3A-8 illustrate various non-limiting embodiments of blades and blade support members; the scalpel-type blades are shown for convenience with generally straight cutting edges similar to a No. 11 scalpel blade. It should be appreciated, however, that the blade may have any suitable straight, curved, angled, or contoured cutting edge geometry, and that the blade support member and associated vapor outlet or outlets may be spaced apart from, and oriented relative to, the cutting edge to deliver condensable vapor V toward tissue adjacent the cutting edge as described herein.

### Vapor Generation Subsystem Configurations

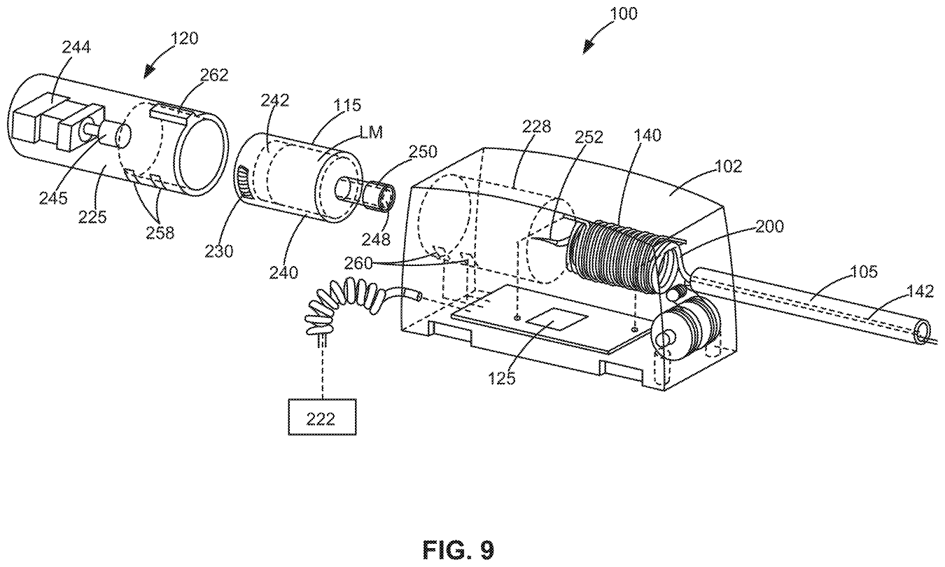

In certain embodiments, the device as shown in FIGS. 1 and 9 includes a vapor generation subsystem configured to convert a controlled flow of a liquid medium LM (e.g., sterile water or saline) into a condensable vapor V for delivery to a treatment site. In some non-limiting embodiments, the vapor generation subsystem comprises a resistive heating assembly or structure 140 configured for heated-wall vaporization, wherein thermal energy is transferred through a wall of metal tubing to vaporize a liquid medium LM flowing through an internal flow channel 142. Such heated-wall configurations may provide a substantially continuous wetted heating surface, which will reduce temperature non-uniformities associated with discrete filament heating elements and will moderate transient thermal fluctuations during initiation or termination of vapor delivery intervals.

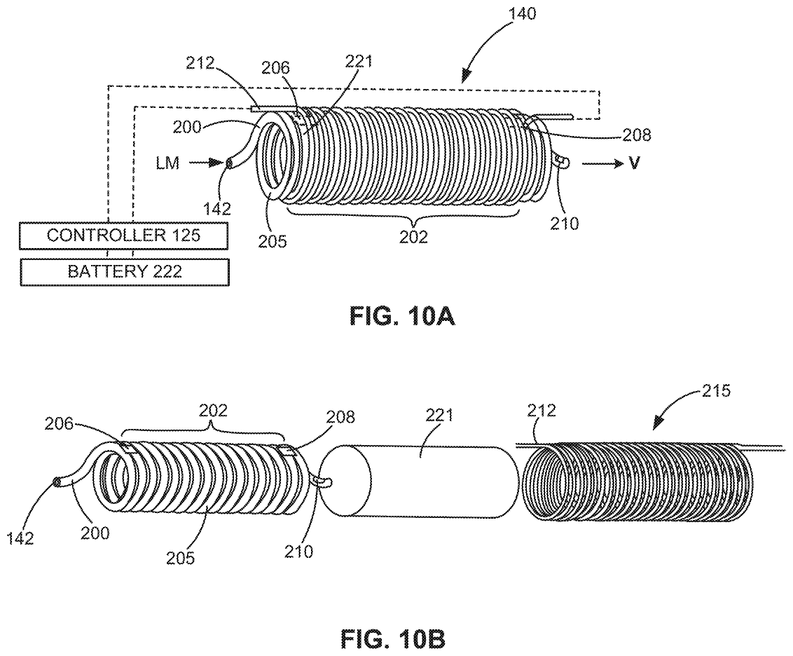

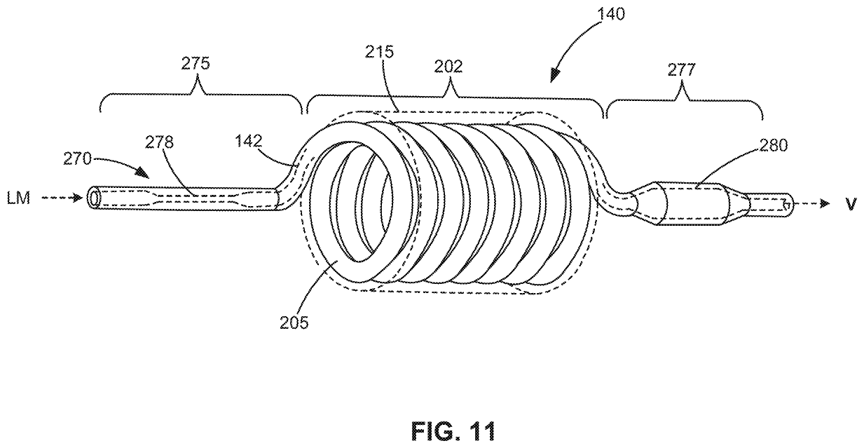

In the variation shown in FIGS. 1 and 9, a first non-limiting configuration of resistive heating structure 140 is provided in a compact form carried in the housing 102. As shown in FIGS. 10A-10B, the heating structure 140 comprises metal tubing 200 defining the flow channel 142 therein extending through a vaporization region 202 where the tubing is formed into a helical coil configuration 205 to provide a compact heated flow path with increased surface area, controlled residence time, and distributed coil wall heating. The helical coil 205 may have an outer diameter of less than about 15 mm, less than about 10 mm, less than about 6 mm, or as small as about 4 mm, and may have an axial coil length of less than about 40 mm, or less than about 30 mm, or less than about 20 mm. The tubing coil 205 may be wound as a single-layer helix or may comprise a concentric multi-layer helix and may include any suitable pitch or turn spacing. In some embodiments, the helical configuration provides a long-heated flow path in a compact volume, which may reduce localized overheating, promote uniform heat flux distribution, and improve repeatability of vapor generation during pulsed operation.

In some embodiments, one or more temperature sensors 206, 208 (FIGS. 10A-10B) are coupled to the tubing coil 205 in the vaporization region 202 to provide temperature signals to the controller 125. For example, two temperature sensors 206 and 208 may be positioned on the coil 205, and a further temperature sensor 210 may optionally be positioned in the flow channel distal to the heating structure to provide additional feedback regarding downstream fluid or vapor temperature.

In the embodiment of FIGS. 1 and 9, electrical energy is delivered to the tubing coil 205 by an external resistive alloy wire 212 formed into a helically wound resistive heating element 215 positioned around the tubing coil 205. The resistive alloy wire may comprise a nickel-chromium-based alloy, such as NiChrome or an alloy such as Kanthal. A suitable resistance alloy for the resistive wire has an electrical resistivity at room temperature ranging from approximately 1.0×10⁻⁶ ohm-meters to approximately 1.5×10⁻⁶ ohm-meters. The resistive alloy wire 212 may have a diameter ranging from 0.005″ to 0.025″ and may be wound at a tight pitch around the tubing coil 205.

In some embodiments, the resistive heating element 215 may be separated from the metallic tubing coil 205 by a thin electrically insulating layer 221, such as a ceramic coating, wash, or sleeve, to prevent electrical coupling while maintaining efficient thermal transfer. The resistive heating element 215 is operatively coupled to the controller 125 and a power source, which may be a remote electrical source, or a remote or onboard battery 222. The heating assembly 140 may further be surrounded by an insulation layer or thermal barrier within the housing 102 to reduce heat loss and improve efficiency. In certain embodiments, the vapor generation subsystem may be configured to vaporize sterile water as a liquid medium LM at a flow rate in a range from about 2 μL/s to about 20 μL/s. By way of non-limiting example, to heat liquid water from approximately 20° C. to approximately 100° C. and vaporize the water at atmospheric pressure, a theoretical energy input of approximately 2.59 kJ per gram of water may be used. Accordingly, vaporizing water at 5 μL/s (≈0.005 g/s) may correspond to a theoretical power of approximately 13 W, and vaporizing water at 10 μL/s (≈0.010 g/s) may correspond to a theoretical power of approximately 26 W. In further embodiments, the electrical power applied to the resistive heating structure may be selected to exceed theoretical vaporization power to account for heat losses, thermal mass, and control margin, such as by operating at about 10-60 W for flows within about 5-15 μL/s, depending on insulation, duty cycle, system pressure, and desired vapor quality or superheat.

In certain embodiments, the vapor heating assembly 140 is configured to vaporize a liquid medium LM comprising sterile water at a flow rate in a range from about 2 μL/s to about 20 μL/s. In some embodiments, vapor V is generated and delivered in a plurality of discrete pulses, wherein electrical power is applied to the resistive heating structure 140 during each pulse to generate condensable vapor, followed by a reduced-power or de-energized interval between pulses. Such pulsed operation may enable rapid initiation and termination of vapor generation with reduced thermal overshoot and improved pulse-to-pulse repeatability while maintaining reliable phase change at low flow rates.

The foregoing dimensions, materials, and power levels are provided as illustrative, non-limiting examples. Other coil diameters, coil lengths, tube sizes, resistive wire geometries, insulating layers, materials, and power levels may be used to achieve a selected vapor output and vapor quality.

In certain embodiments, the vapor generation subsystem may further incorporate flow channel geometries configured to improve vapor quality, suppress intermittent sputter, reduce liquid droplet carryover, and improve repeatability of pulsed vapor delivery. Such flow stabilization and flow-vaporization instability mitigation features are described in greater detail below.

### Cartridge-Based Liquid Medium and Pump Subsystem

FIGS. 1 and 9 illustrate the housing 102 of the vapor-based tissue treatment device 100, wherein the liquid medium LM is carried in a chamber of an insertable cartridge comprising syringe 115. FIG. 9 illustrates a pre-filled syringe cartridge 115 configured to be coupled to a drive housing 225, wherein the assembled cartridge syringe 115 and drive housing 225 are insertable into a receiving channel 228 in the housing 102. An advantage of this cartridge-based variation includes improved options for sterilization and handling of the liquid medium LM, as well as the ability to replace the syringe cartridge 115 during a procedure if the liquid medium LM is depleted. In certain variations, the cartridge syringe 115 may carry a liquid medium LM combined with a pharmacologically active agent that is delivered with thermal vapor V as an adjunct therapy. In such embodiments, multiple insertable cartridges 115 containing different pharmaceutical agents and/or dosages may be provided for selection by the clinician.

In some embodiments, the cartridge syringe 115 is configured for single use and may optionally include an identifier 230 readable by the device to enable, restrict, or parameterize use of the liquid medium LM carried therein. In certain embodiments, the identifier 230 is readable by the controller to verify cartridge compatibility and may comprise, without limitation, a barcode, optical code, resistor, magnetic element, RFID element, or memory chip.

As shown in FIG. 9, the pre-filled syringe cartridge 220 comprises a body 240 defining the syringe chamber that carries the liquid medium LM. A plunger 242 is configured to be advanced by a motor 244 and linear actuator 245 to move the liquid medium LM through a flow channel 142 and toward the resistive heating structure 140. In the embodiment shown, a distal tip 246 of the cartridge syringe 115 includes a discharge outlet 248 sealed by a pierceable seal 250. Upon insertion of the cartridge syringe 115 into the housing 102, the seal 250 may be pierced by a needle 252 to establish fluid communication with the downstream flow channel 142. In certain embodiments, the discharge outlet 248 is sealed prior to use and configured to open automatically upon insertion of the cartridge syringe 115 and drive housing 225 into the housing 102. In some embodiments, opening of the seal 250 irreversibly alters the cartridge to inhibit re-use, and the cartridge syringe 115 is configured to be removed and discarded after a single treatment procedure. In other embodiments, the seal 250 comprises a frangible seal, a burstable seal, or a valve that is opened upon insertion of the cartridge syringe 115 into housing 102. The cartridge syringe 115 may carry a predetermined volume of liquid medium LM ranging from about 2 cc to about 20 cc.

In the embodiment of FIGS. 1 and 9, the drive housing 225 is configured to be insertable and removable relative to the receiving channel 228 in housing 102. The drive housing 225 may carry components of the pump subsystem, such as a DC motor 244 and a linear actuator 245. In some embodiments, an exterior surface of the drive housing 225 includes electrical contacts 258 configured to couple to cooperating electrical contacts 260 in the receiving channel 228 of the housing 102, wherein the cooperating electrical contacts are electrically coupled to the electrical source or battery 222 to provide power to the motor 244. The cartridge syringe 115 and drive housing 225 may further include alignment features configured to ensure proper orientation of the cartridge relative to the drive housing. Similarly, the drive housing 225 may include alignment features, such as key 262 (FIG. 9), configured to ensure proper orientation relative to the receiving channel 228 and to align the electrical contacts 258 and 260.

In certain embodiments, a locking mechanism such as a J-lock, bayonet lock, latch, or other coupling features may be provided to detachably lock the drive housing 225 in the housing 102. In the disassembled view of FIG. 9, the cartridge syringe 115 includes a mechanical interface configured to engage the linear actuator 245.

, such as a lead screw interface, threaded engagement, plunger shaft interface, rack interface, magnetic coupling, or other drive surface. FIG. 1 shows the cartridge syringe 115 and drive housing 225 assembled and inserted into the housing 102 of the device 100.

### User-Adjustable Energy Delivery Control

In certain embodiments, the device includes a user-actuatable control, such as a button, switch, toggle, dial, or touch-sensitive interface, which allows a clinician to select between at least two different energy-delivery settings corresponding to different levels of thermal energy delivered during vapor delivery. In some embodiments, the selected setting corresponds to a desired vapor output, caloric output per unit time, or heating power level delivered by the device during a vapor delivery interval. In a vapor-based device having a handle gripped by the surgeon, the actuatable control may be on the handle. In another variation where the device 100 is coupled to a positioning system or robot, the user-actuatable control may be on a touch screen or other location within the robotic controls.

Actuation of the user-adjustable control provides an input to the controller 125, which adjusts operation of the pump mechanism and electrical power delivered to the resistive heating structure 140 in a coordinated manner based on the selected setting. In particular, selection of a higher energy-delivery setting may cause the controller 125 to increase a flow rate of liquid medium LM delivered toward the heating structure 140 while also increasing electrical current or power delivered to the heating structure 140 to maintain complete or substantially complete vaporization of the liquid medium at the increased flow rate. Selection of a lower energy-delivery setting may cause the controller 125 to reduce the flow rate and correspondingly reduce electrical current or power delivered to the heating structure 140.

In some embodiments, the controller 125 implements predetermined operating profiles, look-up tables, or control algorithms that associate each selectable energy-delivery setting with corresponding liquid-medium flow rates and heating-structure power levels. In other embodiments, the controller dynamically adjusts flow rate and heating-structure current using feedback from one or more sensors, including temperature sensors 208 coupled to the heating structure or a sensor 210 positioned downstream of the heating structure 140 (FIGS. 10A-10B), to maintain vapor generation within a desired operating range for each selected setting. In such embodiments, coordinated adjustment of liquid flow rate and heating-structure power may assist in maintaining stable generation of high-quality vapor while reducing liquid droplet carryover.

The user-adjustable control may allow selection between discrete energy-delivery levels, such as a low setting and a high setting, or among three or more predefined settings. In some embodiments, the controller 125 further controls delivery of vapor in a plurality of discrete delivery intervals or pulses, such as pulses having durations ranging from about 1 second to about 10 seconds, with reduced-power or de-energized intervals between pulses.

### Flow-Vaporization Instability Mitigation Geometries

In certain embodiments shown in FIGS. 11 to 14, the vapor generation subsystem includes one or more flow stabilization features associated with the resistive heating assembly 140 to improve vapor quality and delivery repeatability. In a variation shown in FIG. 11, the resistive heating assembly 140 comprises tube 200 formed into tubular coil 205 with interior flow channel 142 as in the devices of FIGS. 1, 9, and 10A. In FIG. 11, the outer resistive wire wrap 215 is indicated by the dashed line. The heating assembly of FIG. 11 may include an upstream flow restrictor 270, positioned proximal to the heated vaporization region 202 of the flow channel 142 to provide a controlled pressure drop and reduce flow oscillations associated with two-phase vaporization. The term two-phase vaporization as used herein, refers to a condition in which liquid medium LM and vapor V co-exist contemporaneously during a phase-change process in a region of the heated flow channel 142 in the vaporization region 202. In such conditions, the flow may comprise a dynamic mixture of vapor bubbles, bubble growth and collapse, and residual liquid droplets that can result in intermittent pressure fluctuations and variations in vapor fraction.

In a small-diameter heated flow channel 142 as in FIG. 11, such intermittent two-phase behavior may be associated with flow vaporization instability modes, including pressure-drop oscillations, density-wave oscillations, intermittent vapor backflow, and slug or plug flow transitions. Such instabilities may be undesirable in vapor-based tissue treatment devices, as they can reduce vapor quality, increase liquid droplet carryover, and cause sputter or intermittent delivery from a vapor outlet. Accordingly, such instabilities may be mitigated by selected geometrical modifications to an inlet region 275 of a heating structure, the vaporization region 202, and an outlet transition region 277, including restrictions, expansions, staged impedance architectures, diverging segments, manifold-like flow distribution structures, and internal inserts.

In some embodiments, the upstream restrictor 270 functions as a hydraulic damping element that increases backpressure and reduces sensitivity of the vaporization region 202 to inlet compliance, pump pulsatility, and rapid void fraction changes during initiation of boiling. In non-limiting examples, the restrictor 270 comprises a reduced-diameter passage 278, a capillary segment, or orifice having an inner diameter ranging from about 50 μm to about 250 μm, or from about 75 μm to about 200 μm, and an axial length ranging from about 2 mm to about 50 mm, or from about 10 mm to about 20 mm. In certain implementations, such restrictors 270 provide a pressure drop of approximately 1 psi to 20 psi at selected operating flow rates, such as liquid flow rates of about 2 μL/s to about 20 μL/s, thereby damping pressure oscillations and improving repeatability of vapor generation across multiple pulses.

Additionally, or alternatively, the outlet transition region 277 of the heating assembly may be configured with a downstream channel expansion zone 280 distal to the vaporization region 202. In some embodiments, the expansion zone 280 comprises an enlarged cavity or increased-diameter region having an inner diameter ranging from about 1.1× to about 3.0× an inner diameter of the channel 142 in the vaporization region 202, or from about 1.2× to about 2.0×, with an axial length ranging from about 0.5 mm to about 10 mm, or from about 1 mm to about 5 mm. Such an expansion zone 280 may provide a vapor accommodation volume that facilitates vapor disengagement from residual liquid droplets, reduces droplet carryover, and suppresses plug or slug flow transitions prior to vapor exiting the heating assembly.

In certain embodiments, the combination of the upstream restrictor 270 and the downstream expansion zone 280 defines a staged-impedance architecture configured to damp pressure oscillations associated with two-phase vaporization, reduce intermittent vapor backflow, and improve repeatability of high-quality vapor generation prior to delivery toward one or more vapor outlets.

In further variations, the vaporization region 202 may include one or more diverging segments, stepped transitions, microcavities, or distributed surface micropatterns that provide local vapor accommodation volumes and enhance vaporization nucleation to improve vapor quality. In certain embodiments, a diverging segment comprises a diffuser-like expansion having a divergence angle ranging from about 1 degree to about 20 degrees, or from about 2 degrees to about 10 degrees, extending over an axial length ranging from about 1 mm to about 50 mm. In other variations, the micropatterned channel surface may transition to a smooth channel surface.

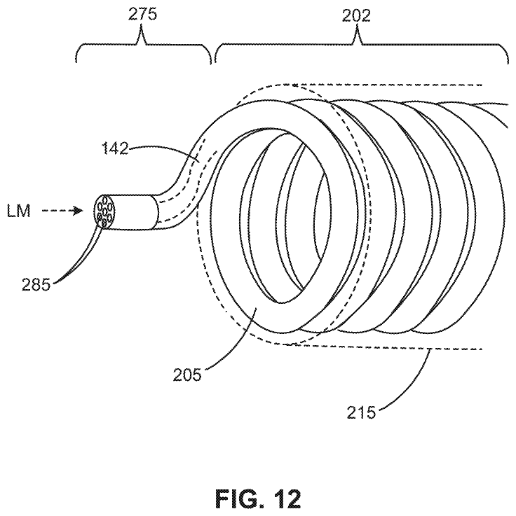

Referring to FIG. 12, in some embodiments, the inlet region 275 proximal to the vaporization region 202 defines a plurality of jets or apertures 285 configured to deliver the liquid medium LM as multiple inlet jets into the flow channel 142. In certain embodiments, the apertures 285 have diameters ranging from about 25 μm to about 300 μm, or from about 50 μm to about 200μm, with a number of apertures ranging from about 2 to about 20, or from about 3 to about 12. The apertures 285 may be arranged circumferentially about a longitudinal axis of the flow channel 142 or in another selected pattern. Such multi-aperture inlet jet configurations may improve liquid distribution, reduce localized stagnation, and suppress rapid bubble coalescence and vapor backflow during boiling onset. In some embodiments, the inlet jets cooperate with the restrictor 270 and/or distal expansion zone 280 describe above to reduce slug transitions and improve pulse-to-pulse repeatability.

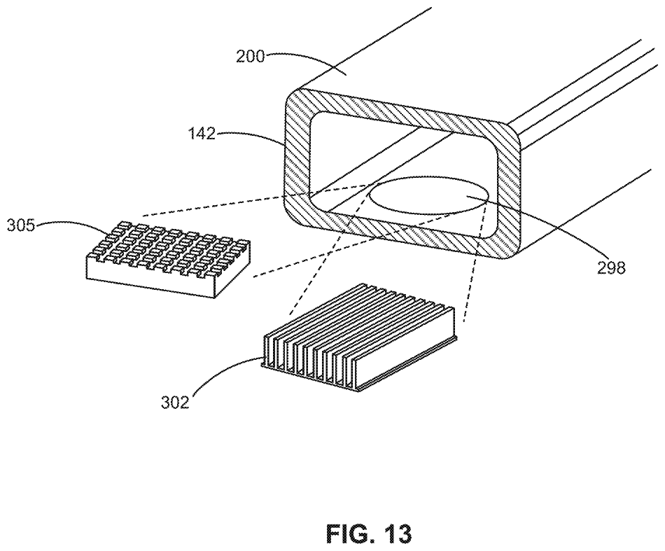

Referring to FIG. 13, in certain embodiments, the heating tube 200 includes an internal insert 298 positioned within the vaporization region of the flow channel 142. The insert 298 may comprise a pin-fin 302 or post array 305 having elements with diameters or cross-sections ranging from about 25 μm to about 500 μm, or from about 50 μm to about 250 μm, with center-to-center spacing ranging from about 50 μm to about 2 mm, or from about 100 μm to about 1 mm. In other embodiments, the insert 298 comprises a porous element having an average pore size ranging from about 10 μm to about 300 μm, or from about 25 μm to about 150 μm. Such inserts 298 may increase effective heat transfer surface area, influence liquid film behavior, promote controlled nucleation, and reduce formation of large vapor plugs by promoting distributed bubble generation and vapor transport. In some embodiments, downstream smooth-wall regions may be provided to facilitate completion of vaporization and reduce droplet entrainment leaving the vaporization region.

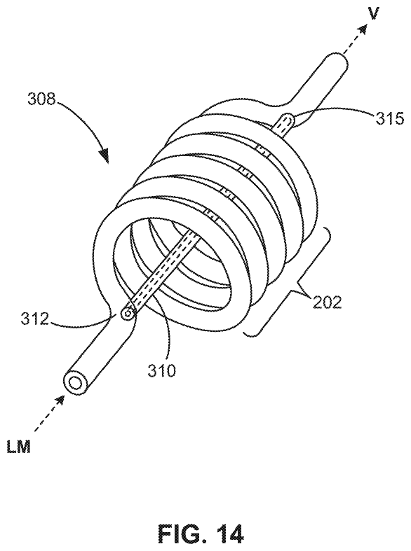

Referring to FIG. 14, in certain embodiments, the heating assembly 308 includes an interconnected manifold or bypass flow passage 310 extending between an upstream junction 312 and a downstream junction 315 relative to the vaporization region 202. In some embodiments, the bypass passage 310 comprises a secondary flow channel having a hydraulic diameter ranging from about 50 μm to about 1 mm, or from about 100 μm to about 500 μm, and an axial length ranging from about 1 mm to about 50 mm, or from about 5 mm to about 25 mm. The bypass passage 310 may provide an alternate flow path for vapor and/or liquid phases to reduce intermittent channel blockage, mitigate pressure spikes associated with vapor slug formation, and improve stability of vapor delivery. In certain embodiments, the bypass passage 310 functions as a vapor relief path or recirculation path that reduces the sensitivity of the vaporization region 202 to rapid void fraction changes.

It should be appreciated that the geometrical mitigation features described with respect to FIGS. 11-14 are provided as non-limiting examples, and variations in restriction ratio, expansion ratio, axial spacing, channel geometry, surface finish, staged heating configuration, and combinations thereof may be selected to reduce two-phase flow instabilities, suppress plug or slug flow transitions, improve vapor fraction, and reduce liquid droplet carryover. Such features may be used individually or in combination in proximal heating assemblies, distal heating assemblies, and hybrid multi-stage vapor generation architectures.

In the blade-based vapor delivery embodiments described herein, including those illustrated in FIGS. 3A to 8, it may be desirable to deliver a repeatable flow of high-quality condensable vapor from one or more vapor outlets 145 positioned at or near the blade to provide consistent thermal treatment adjacent to the cutting edge 112. Accordingly, the flow stabilization and two-phase instability mitigation features described with respect to FIGS. 11-14 may be incorporated into the vapor generation subsystem of the blade-based devices to improve vapor fraction, reduce liquid droplet carryover, suppress sputter or intermittent flow, and enhance delivery repeatability during pulsed operation. In such embodiments, the vapor quality improvements provided by the heating assembly geometries may assist in achieving predictable coagulation, sealing, ablation, or tissue modification at tissue regions adjacent to the cutting edge during cutting or contemporaneously with cutting.

It should be appreciated that any of the blade and vapor outlet configurations described with respect to FIGS. 3A to 8 may be used in combination with any of the vapor generation architectures, flow restrictors, expansion zones, inlet jet structures, inserts, bypass passages, and other geometrical stabilization features described herein, and that such combinations are within the scope of the invention.

### Battery Power Source and Battery-Operated Implementation

In the embodiments of FIGS. 1, 2, and 9, the devices include vapor generation subsystems configured for operation with an onboard power source comprising a battery 222 or battery pack. In such embodiments, the geometry, resistivity, placement, and duty cycle of the vapor-generating resistive heating structure, as well as liquid delivery parameters, may be selected to operate within battery-defined voltage, current, and thermal limits while still achieving effective vaporization of a flow of liquid medium.

In certain variations, the power source comprises a rechargeable battery 222 configured to deliver sufficient electrical power to operate the pump subsystem, controller, and resistive heating structure during vapor generation. By way of example, the battery 222 may be configured to provide peak power levels of approximately 10 Watts to 120 Watts at operating voltages ranging from approximately 3 Volts to 24 Volts, with transient current delivery on the order of 3 Amps to 20 Amps during vaporization cycles. Such power capacity enables multiple vapor delivery cycles per procedure, where vapor delivery intervals or pulses may range from about 1 second to about 10 seconds.

The battery 222 or battery pack may be selected to provide high energy density, compact form factor, and reliable discharge characteristics suitable for the transient current demands of the resistive heating structure. Suitable battery chemistries include, without limitation, lithium-ion (Li-ion), lithium-polymer (LiPo), and lithium iron phosphate (LiFePO₄). In many embodiments, Li-ion batteries are preferred due to their high specific energy, typically in the range of about 150 to 250 Wh/kg, and widespread availability in standardized formats.

Non-limiting examples of suitable Li-ion battery formats include cylindrical cells designated by dimensional codes corresponding to diameter and length in millimeters. In certain embodiments, the device incorporates one or more 18650 cells (approximately 18 mm×65 mm), which provide a balance of capacity (typically 2000-3500 mAh per cell) and compactness, allowing multiple cells to be arranged in series or parallel within the housing or within a removable battery pack. Higher-capacity alternatives include 21700 cells (approximately 21 mm×70 mm, typically 4000-5000 mAh) and 26650 cells (approximately 26 mm×65 mm, typically 4500-5500 mAh) for extended operating time or reduced recharge frequency. For more compact or lower-power embodiments, smaller cylindrical cells such as 16340 cells (approximately 16 mm×34 mm) may be employed, including in disposable or single-use configurations.

In general, the specific battery format may be selected based on desired discharge capability, physical integration within the housing 102 is a variation, and thermal management requirements, rather than on nominal capacity alone. Such standardized battery formats offer high energy density, low self-discharge, and established safety features, including protection against overcharge, over-discharge, and short-circuit conditions, making them well-suited for powering a resistive vapor-generating structure.

The battery 222 or battery pack may be remote from the device or placed directly within the housing and consist of a removable battery pack to allow for charging and rapid replacement. In rechargeable embodiments, the device may include a charging interface, such as a USB-C port, magnetic connector, inductive charging interface, or other suitable coupling. In multi-cell configurations, a battery management system (BMS) may be provided to balance cell charging, monitor temperature and voltage, and ensure safe operation at the required discharge rates.

In certain variations, the controller 125 is configured to inhibit or terminate vapor generation based on one or more battery-related conditions, including insufficient state of charge, excessive discharge current, elevated battery temperature, or voltage outside a predetermined operating range. Such battery-aware interlocks may enhance safety, ensure consistent vapor quality, and prevent incomplete vaporization associated with inadequate power delivery.

The controller 125 may be configured to coordinate the operation of the pump assembly and the resistive heating structure 140 to deliver condensable vapor during one or more delivery intervals or pulses. In some embodiments, the controller allocates an energy budget per pulse and controls both (i) a metered volume of liquid delivered by the pump assembly and (ii) electrical power delivered to the resistive heating structure to satisfy the allocated energy budget while maintaining vaporization of the metered liquid volume.

The battery may define an operating voltage window, discharge current capability, thermal operating range, and state-of-charge characteristics. The device may further include one or more battery condition sensors configured to provide signals indicative of battery voltage, discharge current, battery temperature, state of charge, or combinations thereof. The controller may be configured to control at least one operating parameter of the pump subsystem and at least one operating parameter of electrical power delivered to the resistive heating structure based at least in part on a sensed battery condition.

In some embodiments, the controller 125 is configured to inhibit initiation of vapor delivery or to terminate an ongoing vapor delivery when a sensed battery condition satisfies a threshold condition associated with insufficient available power to maintain vaporization. Such inhibition or termination may occur before or during a delivery interval or pulse.

### Illustrative Power and Battery-Operated Implementation

In one exemplary, non-limiting implementation, a vapor-generating heating structure 140 as shown in FIGS. 1 and 9 was developed to deliver from about 10 cal/sec to about 50 cal/sec. Electrical power was supplied by a rechargeable lithium polymer battery, such as a 14.8-volt battery rated at approximately 450 milliamp-hours with a discharge capability of up to approximately 30 amps. In one example, the battery comprised a Spektrum SPMX4504550 battery developed for high-current applications. Battery voltage supplied to the resistive heating structure was modulated using a pulse-width modulation (PWM) circuit controlled by a microcontroller on a printed circuit board (PCB).

In one example, electrical current delivered to the resistive heating structure was approximately 10 amps, corresponding to approximately 120 watts of electrical power delivered to the heating structure 140 having a resistance of approximately 1.2 ohms. Calorimetric measurements indicated an output of about 25 cal/sec, yielding an overall power conversion efficiency of approximately 88 percent. Battery discharge during a ten-second vapor delivery pulse at 10 amps corresponds to approximately 100 amp-seconds, or approximately 28 milliamp-hours, enabling aggregated vapor delivery durations of about 150 to 180 seconds, which may consist of pulse intervals ranging from about 1 second to about 10 seconds. In a variation, the battery-powered heating structure 140 is configured to deliver vapor medium from a vapor outlet in a working end for at least 60 seconds, at least 120 seconds, or at least 180 seconds.

In an exemplary configuration, the 25 cal/sec vapor delivery corresponded to a liquid medium flow rate of about 6 microliters/sec. Linear actuator speed and force requirements were selected to accommodate nominal operating pressures of approximately 15-20 psi and maximum pressures up to approximately 60 psi. In this example, the linear actuator used current under maximum pressure conditions of less than approximately 0.1 amp, which was negligible compared to the current delivered to the resistive heating structure. The assembly may include additional features such as LED indicators for power and operating state, fault indicators, a compact PCB with an embedded microcontroller, recharging interfaces including wired or inductive charging, and selectable vapor delivery pulse intervals.

The foregoing battery chemistries, voltages, current levels, discharge characteristics, and calorimetric performance values are provided as illustrative, non-limiting examples. Other battery types, battery capacities, operating voltages, current levels, control schemes, and heating structure resistances may be used to provide sufficient energy delivery for vapor generation and tissue treatment consistent with the principles described herein.

### Robotic and Computer-Assisted Positioning of Vapor-Delivery Instruments

In certain embodiments, the vapor ablation systems and vapor-delivery instruments described herein are configured for use with a computer-assisted or robotic positioning system, such as the robotic system illustrated in FIGS. 1, 2, and 9. Such systems may include, without limitation, robotic arms, teleoperated manipulators, computer-controlled positioning stages, motorized advancement mechanisms, powered articulation structures, or other mechanical systems capable of positioning and stabilizing a medical instrument relative to patient anatomy.

In one variation, a robotic positioning system as shown in FIG. 2 is configured to support controlled translation, rotation, articulation, advancement, retraction, and/or stabilization of a vapor-delivery device 100 having a working end 108 configured to deliver thermal energy to tissue via a condensable vapor. In such embodiments, the vapor-delivery instrument comprises the proximal housing carrying a volume of a liquid medium LM and a pump mechanism configured to deliver a controlled flow of the liquid medium to a resistive heating structure to generate a flow of condensable vapor. The condensable vapor is emitted from one or more vapor outlets at or near a working end of the device and delivers thermal energy to tissue through condensation and release of latent heat.

The present disclosure does not require any particular robotic architecture, joint configuration, actuator type, or control topology. Robotic positioning systems suitable for use with the disclosed vapor-delivery instruments include commercially available systems for surgical and interventional procedures, including systems configured for bronchoscopic, endoscopic, laparoscopic, intraluminal, or percutaneous access.

### Coordination of Robotic Positioning and Vapor Delivery

In certain embodiments, a controller 125 is operatively coupled to both a vapor-based device 100 of the type shown in FIG. 1 and the robotic positioning system. The controller 125 may be configured to coordinate positioning of a distal portion of the vapor-delivery device, including a working end with a blade, an elongate catheter, or a working end with jaws or a needle, or combinations thereof, to deliver a condensable vapor to tissue.

In some embodiments, vapor delivery is enabled only when the robotic positioning system has positioned the vapor-delivery instrument at a selected target location, orientation, articulation state, or deployment condition. In other embodiments, vapor delivery is coordinated with robotic advancement, deployment, or stabilization of one or more needles relative to tissue. Such coordination may reduce unintended movement during vapor delivery, improve repeatability of ablation, and enhance safety when delivering vapor adjacent to sensitive anatomical structures.

The controller 125 may coordinate robotic positioning and vapor delivery using clinician input, pre-programmed motion profiles, image-guided navigation data, force sensing, position sensing, articulation feedback, or combinations thereof. The controller may further inhibit, modulate, or interrupt vapor delivery if the robotic system detects deviation from a selected position, force threshold, articulation angle, or deployment state.

The robotic positioning system may be used in conjunction with one or more imaging or navigation modalities, including without limitation endoscopic visualization, ultrasound imaging, fluoroscopy, electromagnetic navigation, optical tracking, bio-capacitance sensing, and/or preoperative imaging registered to patient anatomy.

In some embodiments, imaging or navigation data is used to guide robotic positioning of the vapor-delivery instrument and to select a target location for needle deployment and vapor delivery. In other embodiments, imaging is used to confirm placement prior to vapor delivery and/or to evaluate tissue response following delivery. In certain embodiments, the vapor-delivery instruments described herein are compatible with such imaging and navigation systems because vapor delivery does not require direct electrical coupling to tissue, grounding pads, or large conductive return paths.

The robotic embodiments described herein are illustrative and not limiting. The disclosed vapor-delivery instruments may be used with robotic systems that are fully autonomous, semi-autonomous, teleoperated, or manually assisted. The present disclosure does not require that the vapor-delivery instrument itself include robotic actuators, provided that the instrument is positionable, advanceable, articulatable, and/or stabilizable by an external robotic or computer-assisted system. No particular robotic platform, manufacturer, or control architecture is required, and the disclosed vapor-delivery instruments may be adapted for use with existing robotic systems without departing from the scope of the invention.

### Integration With Blade-Based and Laparoscopic Embodiments

The robotic positioning concepts described above apply to, and are fully compatible with, the blade-based vapor delivery instruments and other vapor-assisted resection systems described herein. In such embodiments, robotic systems may be used to position, articulate, advance, or stabilize an instrument carrying a blade member and one or more vapor outlets, while vapor generation and delivery are performed by the heating, pumping, and control subsystems described elsewhere in this specification.

Accordingly, robotic positioning is disclosed herein as an optional platform-level capability that may cooperate with vapor-based energy delivery across multiple anatomical targets, access routes, and device form factors.

While a number of exemplary embodiments have been described, it will be understood by those of ordinary skill in the art that various modifications, substitutions, and combinations may be made without departing from the spirit and scope of the present disclosure. Methods described herein may be performed in any order of steps that is logically possible, including orders different from those expressly described. In addition, steps or operations may be added, omitted, or combined to achieve desired results.

Unless otherwise indicated, the terms “comprising,” “including,” and “having” are used in an open-ended sense and do not exclude additional elements or steps. Numerical values, ranges, and dimensions described herein are approximate and may vary based on manufacturing tolerances, operating conditions, and design considerations. Features described in connection with one embodiment may be combined with features of other embodiments unless expressly stated otherwise.

Each of the embodiments and variations described herein may include features that are separable from, interchangeable with, or combinable with features of other embodiments. Modifications may be made to adapt a particular device, system, material, composition, or method to specific applications or objectives without departing from the scope of the present disclosure. As used herein, the term “about” refers to variations attributable to manufacturing tolerances, material composition, environmental conditions, and measurement uncertainty as understood by a person of ordinary skill in the art.

References to publications, patents, and patent applications are provided for informational purposes. Any such references are incorporated herein by reference only to the extent consistent with and not limiting of the present disclosure.

The present disclosure is not limited to the particular embodiments described herein but is intended to encompass all modifications, equivalents, and alternatives falling within the scope of the appended claims. The scope of the disclosure further includes variations and embodiments that may become apparent to those skilled in the art in view of this disclosure.

1.-17. (canceled)

18. A vapor ablation device, comprising:
a blade having a sharp cutting edge;
at least one vapor outlet positioned adjacent to but spaced apart from the sharp cutting edge;
a flow channel in fluid communication with the at least one vapor outlet;
a vapor generation system capable of converting a liquid medium into a flow comprising predominantly of a condensable vapor deliverable through the at least one vapor outlet; and
a controller configured to selectively and/or automatically control delivery of the condensable vapor, wherein the blade is configured to cut tissue;
wherein the at least one vapor outlet is configured to direct the condensable vapor to reach tissue adjacent the sharp cutting edge;
wherein the controller is configured to deliver the condensable vapor during or in temporal association with cutting; and
wherein the condensable vapor is configured to condense on the tissue to transfer latent heat to the tissue to provide localized thermal treatment during or contemporaneously with cutting of the tissue.

19. A vapor ablation device, comprising:
a proximal housing carrying (i) a source containing a liquid medium and (ii) a pump assembly;
a working end comprising a blade having a sharp cutting edge;
a blade-adjacent member positioned adjacent to the blade;
a flow channel in fluid communication with the source;
a resistive heating structure thermally coupled to the flow channel and configured to convert a flow of the liquid medium into a flow comprising predominantly a condensable vapor;
at least one vapor outlet carried by, defined by, or formed in the blade-adjacent member and positioned at or near the blade;
wherein the pump assembly is configured to deliver the flow of the liquid medium to the resistive heating structure at a flow rate ranging from about 2 microliters/second to about 20 microliters/second;
a controller configured to operate the pump assembly and the resistive heating structure to generate the flow comprising predominantly condensable vapor; and
wherein the at least one vapor outlet is configured to direct the condensable vapor to reach tissue adjacent the sharp cutting edge during or in temporal association with cutting.

20. The vapor ablation device of claim 19, wherein the condensable vapor is configured to condense on tissue adjacent the sharp cutting edge to transfer latent heat to the tissue.

21. The vapor ablation device of claim 19, wherein the resistive heating structure and the flow rate are configured to generate a flow comprising predominantly condensable vapor delivered from the at least one vapor outlet.

22. The vapor ablation device of claim 19, wherein the at least one vapor outlet is spaced apart from the sharp cutting edge such that the at least one vapor outlet is positioned outside a primary tissue-contact region of the blade during cutting.

23. The vapor ablation device of claim 19, wherein the controller is configured to coordinate (i) the flow rate of the liquid medium and (ii) power delivered to the resistive heating structure to maintain generation of the condensable vapor.

24. The vapor ablation device of claim 19, wherein the controller is configured to initiate and/or terminate delivery of the condensable vapor in response to user input.

25. The vapor ablation device of claim 19, wherein the controller is configured to deliver the condensable vapor in discrete pulses.

26. The vapor ablation device of claim 19, wherein the condensable vapor is delivered to at least one side of the blade to treat tissue along a cut path.

27. The vapor ablation device of claim 19, wherein the condensable vapor is delivered to opposing sides of the blade to treat tissue on both sides of the cut path.

28. The vapor ablation device of claim 19, wherein the at least one vapor outlet defines an outlet axis oriented to direct the condensable vapor toward the sharp cutting edge.

29. The vapor ablation device of claim 19, wherein the condensable vapor modifies tissue properties adjacent the sharp cutting edge to reduce bleeding.

30. The vapor ablation device of claim 19, wherein the blade is removably coupled to the working end such that the blade is replaceable.

31. The vapor ablation device of claim 19, further comprising an aspiration port positioned in the working end.

32. The vapor ablation device of claim 19, wherein the resistive heating structure comprises a resistive element in a helical form disposed within or about at least a portion of the flow channel.

33. The vapor ablation device of claim 19, wherein the resistive heating structure has an overall electrical resistance of less than about 5 ohms.

34. The vapor ablation device of claim 19, wherein the resistive heating structure comprises a resistance alloy having an electrical resistivity at room temperature ranging from approximately 1.0×10⁻⁶ ohm-meters to approximately 1.5×10⁻⁶ ohm-meters.
